(19) United States
(12) Patent Application Publication
(10) Pub. No.: US 2009/0087913 A1
(43) Pub. Date: Apr. 2, 2009

(54) Analysis of conjugated metabolites of alcohol consumption

(75) Inventor: Takeo Sakuma, Richmond Hill (CA)

Correspondence Address: Ralph A. Dowell of DOWELL & DOWELL P.C., 2111 Eisenhower Ave, Suite 406, Alexandria, VA, 22314, US

(73) Assignees: MDS Analytical Technologies, a business unit of MDS Inc.,doing business through its Sciex Division, Concord (CA); Applied Biosystems Inc., Farmingham, MA (US)

(21) Appl. No.: 12/285,024

(22) Filed: Sep. 29, 2008

Related U.S. Application Data
(60) Provisional application No. 60/976,539, filed on Oct. 1, 2007.

(51) Int. Cl. G01N 33/98; C12Q 1/02; G01N 30/72; C12M 1/34; G01N 37/00; G01N 33/70; G01N 35/00

(52) U.S. Cl. 436/43; 436/95; 436/119; 435/29; 436/173; 436/132; 422/82.05; 422/61; 435/288.7

(57) ABSTRACT

A method, system, kit and uses for quantifying and normalizing at least one product of ethanol metabolism are provided. A method is provided for quantifying and normalizing at least one product of ethanol metabolism in a sample comprising creatinine. The method comprises adding a predetermined amount of at least one internal standard to the sample; adding deuterated creatinine to the sample; detecting and measuring at least one product of ethanol metabolism, the predetermined amount of at least one internal standard in the sample, the deuterated creatinine, and the creatinine. The method also comprises quantifying the amount of at least one product of ethanol metabolism in the sample using the measurement of the at least one internal standard; quantifying the amount of creatinine in the sample using the measurement of the deuterated creatinine; and normalizing the quantity of the at least one product of metabolism using the measurement of the creatinine.

### RELATED APPLICATION

This application claims priority from U.S. Provisional Patent Application Ser. No. 60/976,539 filed Oct. 1, 2007, the contents of which are incorporated herein by reference.

### FIELD

The applicant's teachings relate to a method of quantifying and normalizing products of ethanol metabolism in a sample.

### INTRODUCTION

Detection and quantification of metabolites in a sample obtained from a source can provide information about substances present in the source.

### SUMMARY

In accordance with an aspect of the applicant's teachings, there is provided a method of quantifying and normalizing at least one product of ethanol metabolism in a sample comprising creatinine. The method comprises adding a predetermined amount of at least one internal standard, adding deuterated creatinine to the sample, detecting and measuring at least one product of ethanol metabolism, the predetermined amount of at least one internal standard in the sample, deuterated creatinine, and creatinine. The method also comprises quantifying the amount of at least one product of ethanol metabolism in the sample using the measurement of at least one internal standard, quantifying the amount of creatinine in the sample using the measurement of the deuterated creatinine, and normalizing the quantity of at least one product of ethanol metabolism using the measurement of creatinine.

In another aspect, there is provided a system for monitoring ethanol metabolism in a source using a mass spectrometer to analyze a sample from the source. The sample comprises creatinine which can be indicative of the physical state of the source. The system comprises a controller adapted to automatically dilute the sample by a predetermined amount at least once; add a predetermined amount of an internal standard to the at least one diluted sample; add deuterated creatinine to the sample; detect and measure at least one product of ethanol metabolism, at least one internal standard in the sample, deuterated creatinine, and creatinine; quantify the amount of at least one product of metabolism in the sample using the measurement of at least one internal standard; quantify the amount of creatinine in the sample using the measurement of the deuterated creatinine; and normalize the quantity of at least one product of ethanol metabolism using the measurement of creatinine.

In accordance with another aspect of the applicant's teachings, there is provided a kit for quantifying and normalizing at least one product of ethanol metabolism in a sample comprising creatinine. The kit comprises at least one of the following: a sample, a deuterated internal standard, a calibration standard, a quality control check, instructions, and combinations thereof.

### BRIEF DESCRIPTION OF THE FIGURES

The skilled person in the art will understand that the drawings, described below, are for illustration purposes only. The drawings are not intended to limit the scope of the applicant's teachings in any way. Like references are intended to refer to like or corresponding parts, and in which:

FIG. 1 compares diluted urine matrix calculated concentrations with calculated concentration of samples in a standard matrix.

FIG. 2 shows the structures of six analytes.

FIGS. 3 and 4 describe the automated calibration solution preparation pre-treatment method.

FIGS. 5 and 6 schematically illustrate the dual column plumbing configuration.

FIG. 7 schematically illustrates the 10-port valve configuration.

FIGS. 8 and 9 show the standard drink amounts in various countries.

FIG. 10 shows the production of metabolites over time after consumption of beer and red wine.

FIG. 11 shows the production of metabolites over time after consumption of Brazilian rum.

FIG. 12 shows the production of metabolites over time after consumption of Polish lager beer.

FIG. 13 shows the production of metabolites over time after consumption of Italian red wine.

FIGS. 14, 15, and 16 show examples of the variation of creatinine with different volumes of urine and measured metabolite concentrations.

### DESCRIPTION OF VARIOUS EMBODIMENTS

According to various embodiments of the applicant's teachings, a method for quantifying and normalizing at least one product of ethanol metabolism in a sample comprising creatinine is provided. The method can comprise adding a predetermined amount of at least one internal standard to the sample, and adding deuterated creatinine to the sample. The method can comprise detecting and measuring the at least one product of ethanol metabolism, the at least one internal standard in the sample, the deuterated creatinine, and the creatinine. The method can comprise quantifying the amount of the at least one product of ethanol metabolism in the sample using the measurement of the at least one internal standard, and quantifying the amount of creatinine in the sample using the measurement of the deuterated creatinine. The method can comprise normalizing the quantity of the at least one product of ethanol metabolism using the measurement of the creatinine.

According to various embodiments of the applicant's teachings, the sample can be obtained from a source, such as a mammal. For example, the mammal can be a human, a primate, or other lab animals and the sample can be urine, saliva, milk, blood, or other biological fluids and tissues. Samples such as milk, blood, or other biological fluids and tissues can be pre-treated to remove lipids and proteins before use in the applicant's method.

According to various embodiments of the applicant's teachings, the product of metabolism can be a metabolite of ethanol, for example, which can be indicative of ethanol present in the source. The product of metabolism can be a conjugated version of the substance present in a source. For example, if a source, such as a mammal, consumed ethanol, the product of metabolism can be ethyl sulphate and/or ethyl glucuronide.

According to various embodiments of the applicant's teachings, the detection and measurement conducted in various embodiments of applicant's teachings can be conducted using, for example, a mass spectrometer, such as, for example, a mass spectrometer comprising a triple quadrupole. Other types of mass spectrometer including various types of Ion Traps, Linear Ion Traps, Time of Flight analyzers, magnetic sector instruments all of which could also be used.

According to various embodiments of the applicant's teachings, the components of the sample can occur at varying concentrations as a result of the “thickness” or concentration of the sample. For example, the thickness of urine can reflect, for example, the source's physical state; for example, the thickness can reflect the amount of physical activity, the fluid consumption, the salt intake, muscle mass, or kidney function of the source. Certain components in the sample, such as creatinine or hydrocortisone, can be indicative of the source's physical state. The sample may comprise urine, blood or plasma. These components can be used to normalize the detected amounts of metabolites. Normalization of the detected amounts of metabolites can produce a more accurate quantification of the metabolite.

According to various embodiments of the applicant's teachings, at least one internal standard can be added to the sample before analysis of the sample. An internal standard can comprise a known quantity of a chemical having a chemical structure that mimics the chemical structure of a component of interest. The chemical of the internal standard can comprise an additional component which can be detectable by whichever mode of detection is used. For example, at least one hydrogen atom of the structure could be replaced with a deuterium atom, which allows for detection by mass spectrometry separately from the chemical that it mimics. Preferably, multiple deuterium atoms can be used. Quantification of the known quantity of the chemical of the internal standard can be used to identify and/or quantify a component of interest.

According to various embodiments of the applicant's teachings, the internal standards can be added manually or automatically by, for example, as an HPLC pre-treatment method. The internal standards can be diluted, for example, they can be serially diluted, either manually or automatically, by, for example, an HPLC method. The internal standard can comprise a chemical having a chemical structure that mimics that of a component in the sample. For example, the chemical can have a structure which mimics creatinine, hydroxycortisone, ethyl sulphate, or ethyl glucuronide. The chemical of the internal standard can be modified to be identified, detected, and/or quantified. For example, if a mass spectrometer is being used with the method, the chemical can be deuterated. Thus, the internal standards can comprise deuterated creatinine, deuterated hydroxycortisone, deuterated ethyl glucuronide, and/or deuterated ethyl sulphate.

The methods according to various embodiments of applicant's teachings can comprise at least one dilution, or serial dilutions, of the sample, either before and/or after the addition of an internal standard. The dilutions can be done manually and/or automatically. According to various embodiments of the applicant's teachings, the methods can be automated. For example, automated dilution of urine samples and automated preparation of a calibration curve sample set.

The methods according to various embodiments of applicant's teachings can be used to predict the time and level of alcohol in a source, such as a mammal, consumed as an alcoholic beverage, for example. According to various embodiments of the applicant's teachings, the methods can be used to monitor alcohol in a source, such as a mammal.

According to various embodiments of applicant's teachings, a system for monitoring ethanol metabolism in a source is provided. The system can include the use of a mass spectrometer to analyze a sample from the source. The sample can comprise creatinine indicative of the physical state of the source. The system can comprise a controller adapted to automatically dilute the sample by a predetermined amount at least once. The controller can be adapted to add a predetermined amount of an internal standard to the at least one diluted sample, and adapted to add deuterated creatinine to the sample. The controller can be adapted to detect and measure at least one product of ethanol metabolism, the at least one internal standard in the sample, the deuterated creatinine, and the creatinine. The controller can be adapted to quantify the amount of the at least one product of ethanol metabolism in the sample using the measurement of the at least one internal standard. The controller can be adapted to quantify the amount of creatinine in the sample using the measurement of the deuterated creatinine and adapted to normalize the quantity of the at least one product of ethanol metabolism using the measurement of the creatinine.

According to various embodiments of applicant's teachings, a kit of parts may be provided for quantifying and normalizing at least one product of ethanol metabolism in a sample that comprises creatinine. The kit comprises at least one of the following: a sample, a deuterated internal standard, a calibration standard, a quality control check, and combinations thereof. Typically, quality control checks can be made with predetermined low, medium, and high concentration solutions to produce certain ion counts.

Aspects of the applicant's teachings may be further understood in light of the following examples, which should not be construed as limiting the scope of the applicant's teachings in any way.

### EXAMPLE 1

The method used for this example detected six chemical species in less than four minutes: (1) ethyl glucuronide and (2) ethyl sulphate, conjugated metabolites of ethyl alcohol consumption in urine and their d5-deuterated internal standards, creatinine, an indicator for the “thickness of urine”, and d3-deuterated creatinine as an internal standard. These metabolite concentrations were normalized to 1 g creatinine/L urine

For example, calibration solutions were automatically prepared by serially diluting a stock solution of mixed standards in urine or in a solvent at 1:1 using a custom-configured Shimadzu pre-treatment program. Because urine can suppress ethyl glucuronide signals spiked standard solutions in undiluted urine give approximately 1/10 to 1/15 signals when compared to those in solvent only. However, 1:10 dilution restores the original signal. For this reason it was necessary to dilute the samples to reduce the matrix effect. Urine samples were treated as follows:

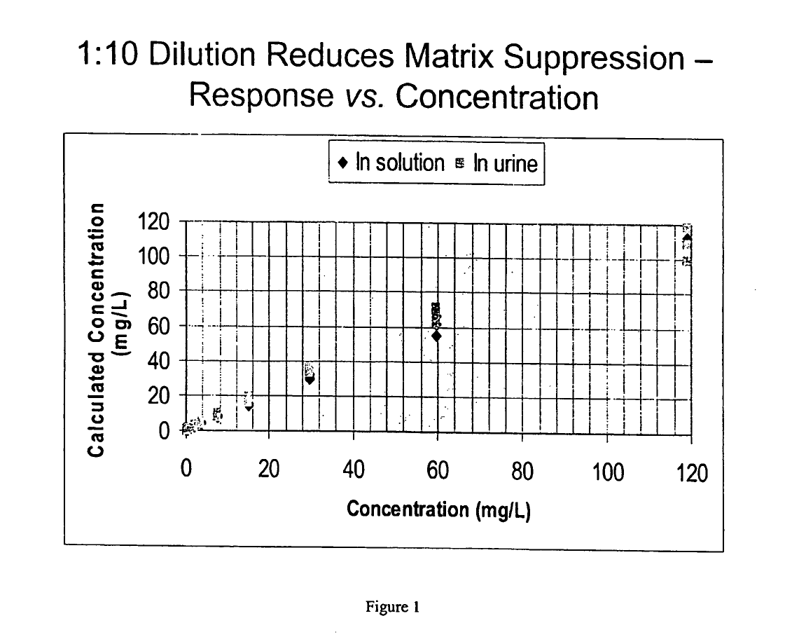

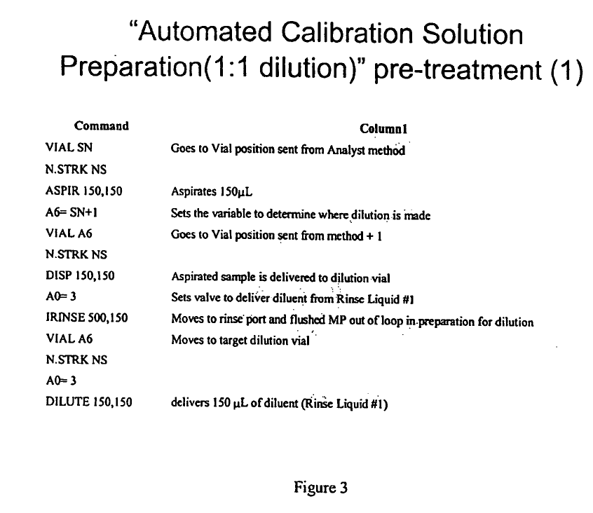

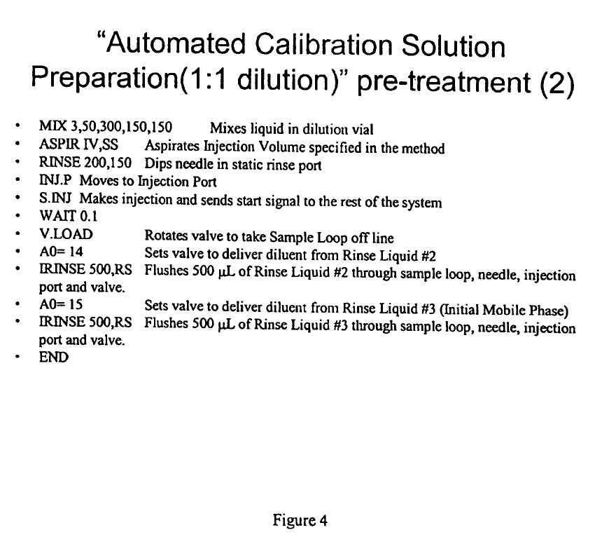

Each urine sample (100 μL) was mixed with 200 μL of a solution (80% water+20% acetonitrile) containing internal standards and 700 μL of acetonitrile using a pre-treatment program as described in FIGS. 3 and 4 thus minimizing human error and possible contamination. FIG. 1 shows a 1:10 dilution reduces matrix suppression-response vs. concentration. If there was matrix suppression, the diluted urine matrix calculated concentrations (pink) would fall below the calculated concentration of samples in a standard matrix (blue)—that was not the case in this experiment, and hence the amount of dilution is used is reasonable in analysis.

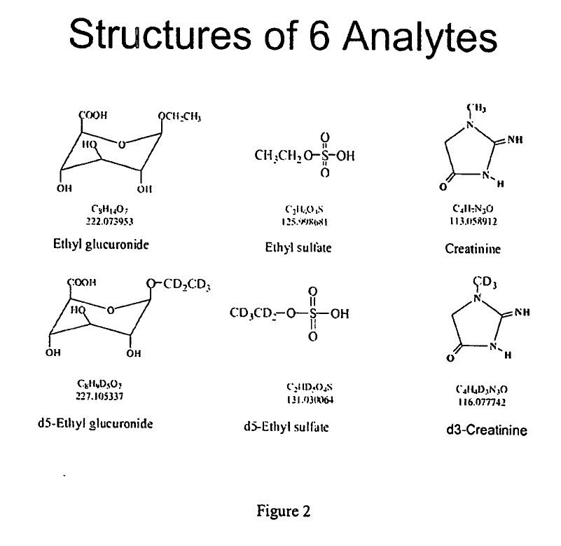

The amounts of ethyl glucuronide and ethyl sulphate were adjusted to that of creatinine (100 mg/dL or 1000 mg/L) as per “Forensic Confirmatory Analysis of Ethyl Sulphate—A New Marker for Alcohol Consumption—by Liquid Chromatography/Electrospray Ionization/Tandem Mass Spectrometer” S. Dresen, W. Weinmann, and F. M. Wurst, J. Am. Soc. Mass. Spectrom., 2004, 15, 1644-1648. In this paper, the metabolites were normalized to creatinine, but the creatinine was measured using an alternative technique, whereas in the applicant's teachings, the creatinine was measured at the same time as the metabolites using the same LC-MS/MS run. FIG. 2 shows the structures of six analytes.

Instruments used for this study include a Shimadzu Prominence, SIL-HT Dual Gradient System consisting of 1×CBM-20A controller, 4×LC-20AD pumps, 1×SIL-20AC auto sampler, 1×CTO-20AC column oven with 2×FCV-20AH2 valves, and 1×DGU-20A3 on-line degasser. An additional pump, LC-10ADvp, and a degasser, DGU14A were used to deliver a solvent to the MS source, while salts were being dumped from the line. The mass spectrometer employed for this study was an API-3200™ triple quadrupole system, operated under multiple reaction monitoring mode (MRM), where a series of precursor and unique fragment ion pairs were monitored one after another in a rotating order. A minimum of 2 ion pairs were monitored per chemical species as per a European GLP Guideline, “Commission Decision of 12 Aug. 2002 implementing Council Directive 96/23/EC concerning the performance of analytical methods and the interpretation of results”, Official Journal of the European Communities, L221/12 17.8.2002, 2002/657/EC, for forensic MS/MS applications.

Reagents

Creatinine was available from Sigma-Aldrich, St. Louis, Mo., USA” P/N C-4255 (http://www.sigmaaldrich.com). D3-creatinine was available from C/D/N Isotopes, Pointe-Claire, Quebec, Canada: P/N D-3689 (http://www.cdnisotopes.com). Ethyl glucuronide (d0 and d5) were available from Cerilliant Corporation, 811 Paloma Drive, Suite A, Round Rock, Tex. 78664, USA. Ethyl sulphuric acid sodium salt was available from Tokyo Kasei Kogyo company Ltd., 6-15-9 Toshima, Kita-ku, Tokyo, Japan (E0277). D5-ethyl sulphate was synthesized by adding d5-ethanol (C/D/N Isotopes Inc., P/N:D-108 116 μL, 1.96 mM) to sulphuric acid (Sigma-Aldrich, #380075, 106 μL, 1.93 mM) in a reacti-vial and heated at 80° C. for 60 minutes. It was diluted to 1 mg/mL in water, and used to prepare a standard solution. Ammonium formate was available from Sigma-Aldrich (product #F-200 Formic acid was available from EMD (AnalaR(R), 98-100%, product # B10115). Acetonitrile (BAKER ANALYZED(R) 9017-03) was obtained from J T Baker. Millipore Q 18MΩ deionized water was used.

HPLC Method

A dual-column liquid chromatography system was used to realize high throughput analysis. The diverter valve attached to the mass spectrometer was also used to divert the early and late LC eluents to waste, while a fifth pump sent a clean solvent to the MS.

Mobile phases A, B, C, D, and Rinse 3 solution comprised 70% acetonitrile+30% water+10 mM ammonium formate, pH adjusted to 5.0 with a small amount of formic acid at a flow rate of 0.35 mL/min (isocratic). Pump 5 used the same composition. Rinse 1 comprised 80% water+20% acetonitrile+500 ng/mL d5-ethyl glucuronide+100 ng/mL d5-ethyl sulphate+1,500 ng/mL d3-creatinine. Rinse 2 comprised acetonitrile (100%). The column was a Waters Atlantis (R) HILIC (Waters, Milford, USA) silica 3 micron, 3.0×100 mm with a matching guard column, heated at 50° C.

FIGS. 3 and 4 show the automated calibration solution preparation (1:1 dilution) pre-treatment method.

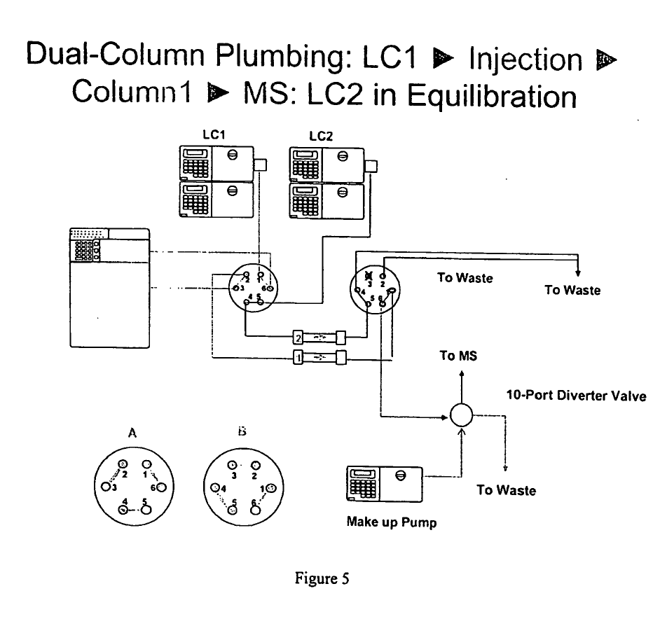

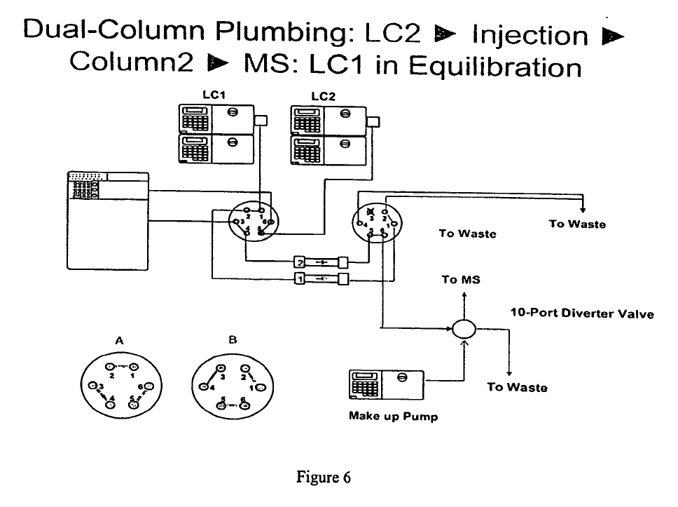

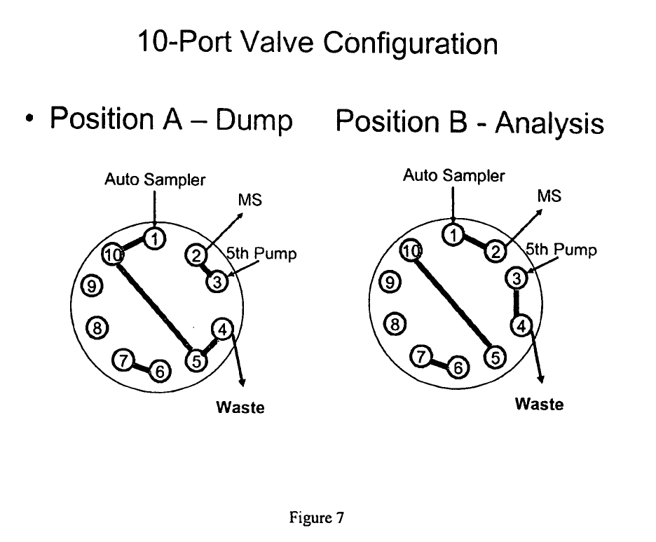

FIGS. 5-7 show the plumbing configuration such that the sample can be automatically injected onto column 1 or 2 (FIGS. 5 and 6 respectively) and the valve configuration can allow the sample to be diverted and the flow replaced by acetonitrile at times when the compound is not eluting but the urine matrix is.

Using a Shimadzu Prominence system and a standard 70-vial tray, the auto sample dilution pre-treatment shown in Table 1 are automatically done. This program can be changed to use a 105-vial tray or 175-vial tray.

Alcohol Consumption Experiments—Background Readings

The determination of metabolites of alcohol can be used as an indicator of alcohol consumption, typically through consumption of alcoholic beverages. Certain other food, medicines and appliances contain alcohol that if also used could potentially become metabolites and increase the reading over and above that derived from alcoholic beverages. In order to determine how alcohol-containing medications and desserts will affect the readings for ethyl glucuronide (Et-G) and ethyl sulphate (Et-S), volunteers were asked to use (1) alcoholic gel to disinfect hands at a hospital, (2) Robitussin® cough syrup, (3) mouthwash, (4) Tiramisu cake, (5) face cleansing cloth, (6) sherry trifle (7) Irish coffee 1 measure liquor in a creamy coffee), (8) a red wine used to cook meat and (9) ham with beer glaze, all at normal usages.

Urine samples were collected before and after the use or consumption. Except for Robitussin, no measurable amounts of Et-G or Et-S were found in the urine samples of the volunteers. Urine samples collected 2 and 7 hours after taking Robitussin showed an increase in Et-S, but not Et-G.

Therefore, it is unlikely that this method will produce false-positive readings, as long as the amount of consumption is reasonable.

Alcohol Consumption Experiments

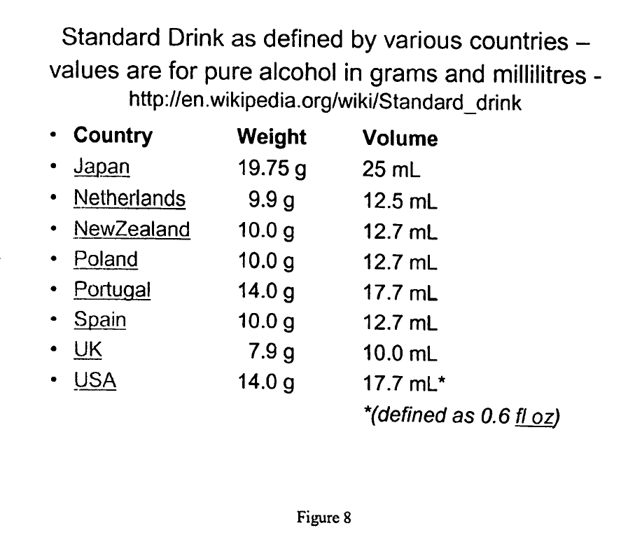

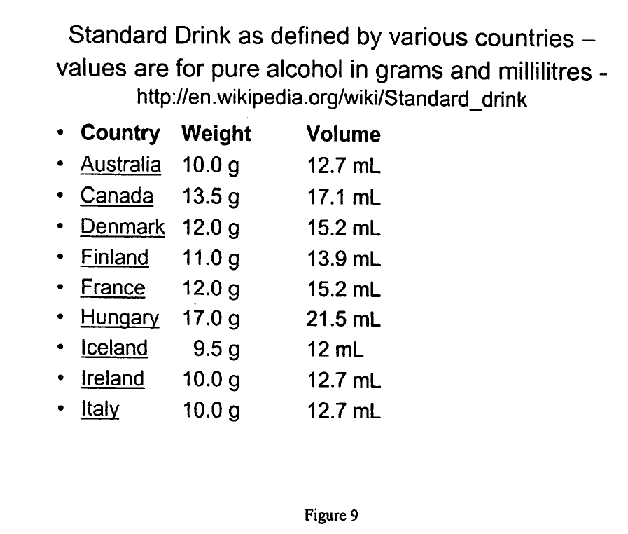

Standard drink amounts in various countries are shown in FIGS. 8 and 9. In order to simulate various consumption scenarios by airline pilots, machine operators, patients undergoing an alcohol withdrawal program, volunteers were asked to consume the following drinks with meals. The selection of meals was left to the discretion of each volunteer.

(1) Red French wine (250 mL, 12% alcohol content)+Portuguese red wine (100 mL, 17.5%) consumed by a female volunteer.

(2) One bottle of Canadian lager beer (355 mL, 5%)+Ontario red wine (1.2 L, 13.5%) consumed by a female volunteer.

(3) 2 Bottles of Ontario lager beer (710 mL, 5%)+Ontario white wine (1.2 L, 13.5%) consumed by a male volunteer.

(4) Polish lager beer (Zywiec, 5.5%, IL) consumed by a male volunteer.

(5) One can of Asahi lager beer (500 mL, 5%)+400 mL Gekkeikan Japanese sake (400 mL, 16%) consumed by a male volunteer.

(6) Appleton white Jamaican Rum (20%, 180 mL over 2 hours) consumed by a male volunteer.

(7) French red wine (ca. 500 mL, 12%) consumed by a male volunteer.

(8) Pedra 90, Brazilian Rum (100 mL, 39%) consumed by a male volunteer.

(9) English Gin (50 mL, 40%) consumed by a male volunteer.

(10) Port wine (100 mL, 18%) consumed by a female volunteer.

(11) Chinese glutinous rice wine (400 mL, 14%) consumed by a male volunteer.

(12) English-made Guinness beer (600 mL, 5%) consumed by a male volunteer.

(13) Bailey's Irish Cream on ice (ca. 300 mL, 17%) consumed by a male volunteer.

(14) Single Malt Scotch Whiskey (60 mL, 40%) consumed by a male volunteer.

(15) Tequila (125 mL, 40%) consumed by a female volunteer.

Urine samples were collected before and after consumption of alcohol beverage.

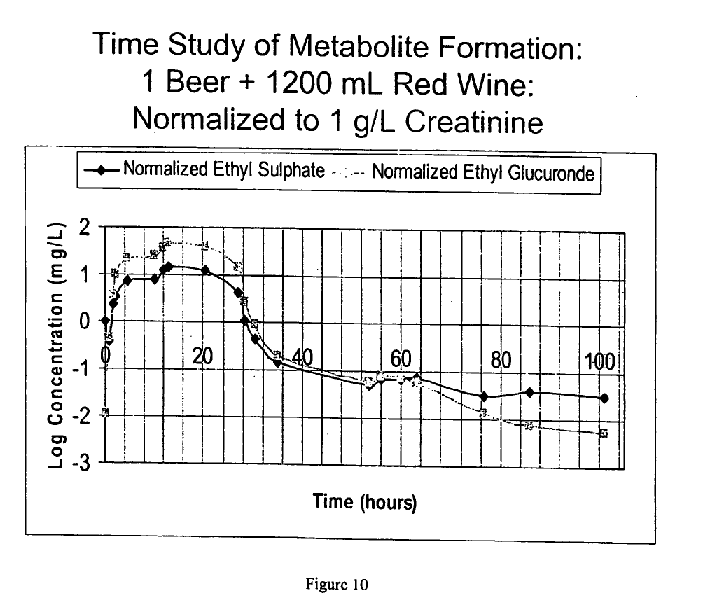

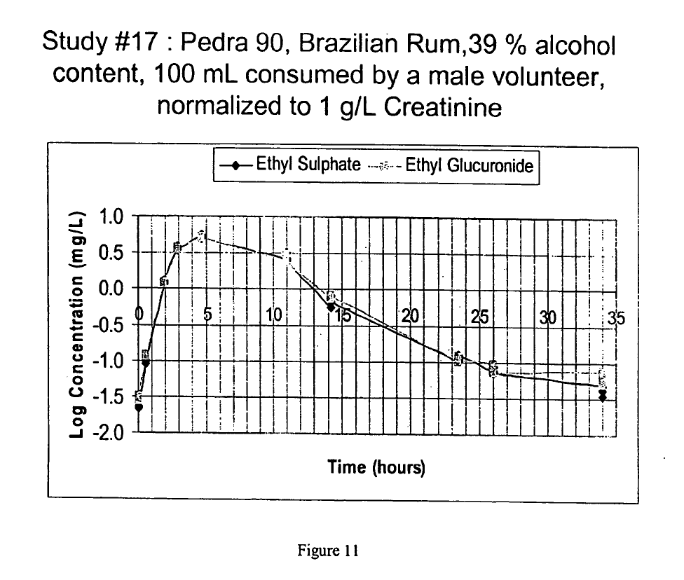

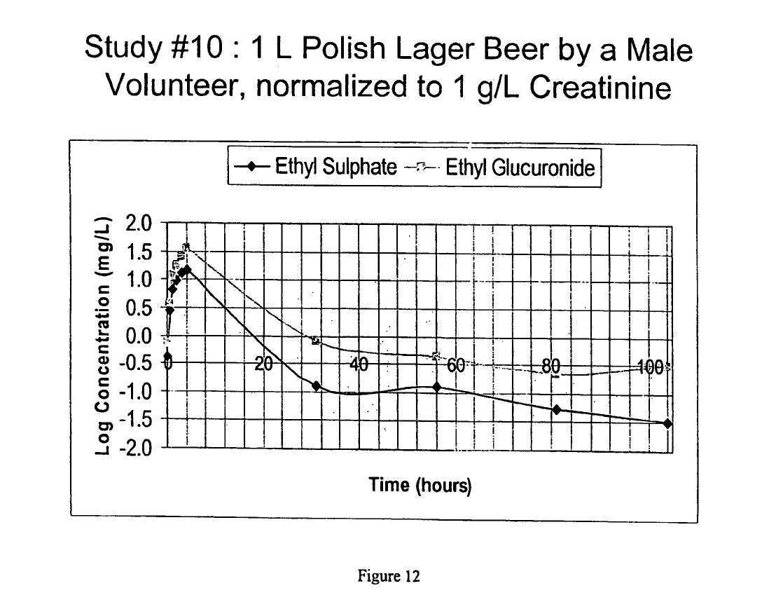

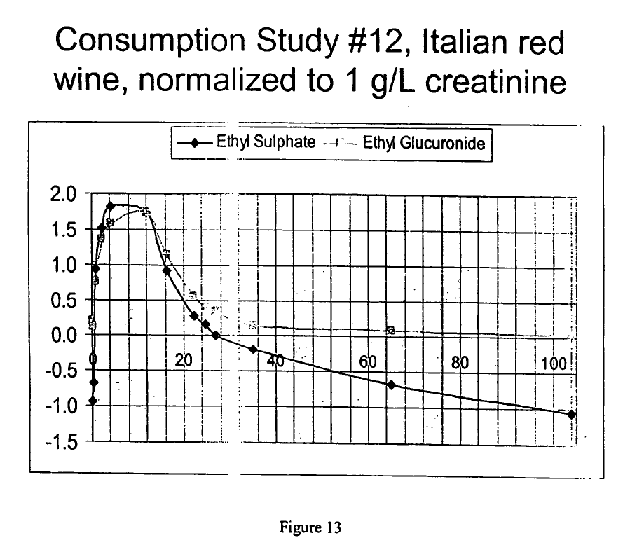

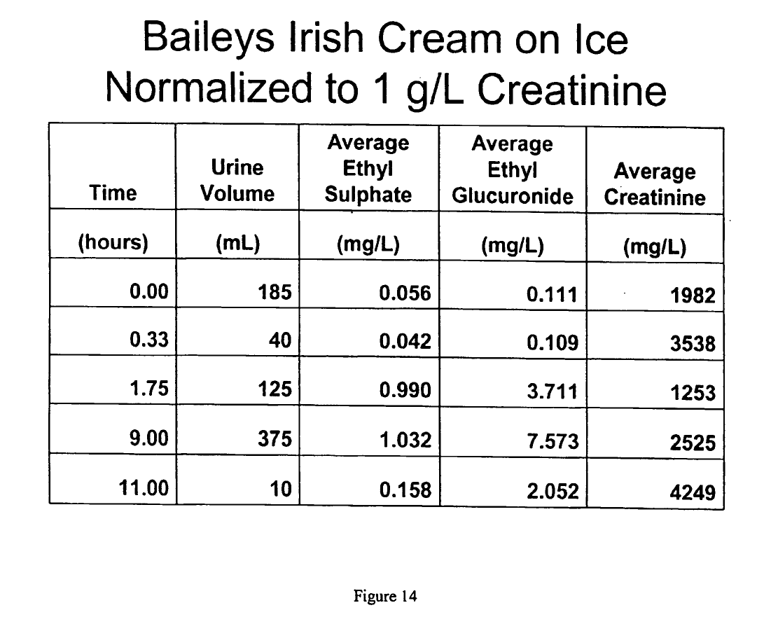

Volumes were recorded and a portion of urine was kept in a 15-mL centrifuge tube at 4° C. for LC/MS/MS analysis. Samples were analyzed as above and plotted as concentration of metabolites of ethanol (sulphate and glucuronide) in urine over time. This shows the production of the metabolites over time after consumption. FIGS. 10-13 show such curves for selected cases. It is shown that the concentration of metabolites in urine increases measurably immediately after consumption, and returns to normal at least 20 hours after consumption. The elevated level of the metabolite is indicative of consumption. The method, which normalizes the concentration to creatinine, shows good agreement between the decay curves of ethyl sulfate and glucuronide. FIG. 14 shows the variation of creatinine with different volumes of urine and measured metabolite concentrations.

While the measurement of urinary concentration of metabolites of ethanol reveals elevated levels post-consumption, in order to relate this concentration to consumption volume it is necessary to perform a mass balance of the metabolite normalized to urinary output and also to the quantity of metabolite formed from the total ethanol ingested.

To evaluate the proportion of ethanol metabolized the mass balance was studied. In one case, 101 hours after the consumption of a beer and red wine (141.8 g ethanol), more than 23.84 mg of ethyl sulphate and 72.86 mg of ethyl glucuronide were formed and discharged.

Stoichiometry is as follows:

$${C}_{2} {H}_{5} {OH}_{46} + {H}_{2} S {{O}_{4}}_{98} -> {C}_{2} {{H}_{5} O}_{126} {SO}_{3} H$$

$$46 \text{:} 126 = 141.8 ( g ) \text{:} X$$

$$\begin{matrix}X = ( 126 \text{/} 46 ) \times 141.8 ( g ) \\ = 388.4 ( g ) ( 0.02384 g ) \text{/} ( 388.4 g ) \times 100 \\ = 0.00614 ( \% )\end{matrix}$$

Similarly, for ethyl glucuronide

46:222=141.8 (g):Y Y=(222)/(46)×141.8 (g)=684.3 (g) (0.07286 g)/(684.3 g)×100=0.0106(%)

Calculations show that 0.0061% of ethanol was converted into ethyl sulphate, and 0.0106% of ethanol was converted into ethyl glucuronide and discharged into the urinary system. It is said the majority of alcohol is converted into carbon dioxide and water.

In another case, a female volunteer consumed French red wine (250 mL, 12%) and Portuguese port wine (100 mL, 17.5%) in 30 minutes or so.

The total amount of alcohol consumed was 37.478 grams. Urine samples were collected over 46.45 hours, volume of each discharge was measured and recorded.

Alcohol introduced: 250 mL×12(%)/100×0.789 (g/mL)+100 mL×17.5 (%)/100×0.789 (g/mL)=37.48 grams ethanol

11.091 mg ethyl sulphate detected . . . 0.010%

The importance of normalization was illustrated when a male volunteer consumed 1 can of chilled Asahi Super Dry beer (500 mL, 5% ) followed by warm Gekkeikan Sake (400 mL, 16%) in approximately 2 hours.

The total ethanol introduced to his system was 500×0.05×0.789+400×0.16×0.789=70.211 g.

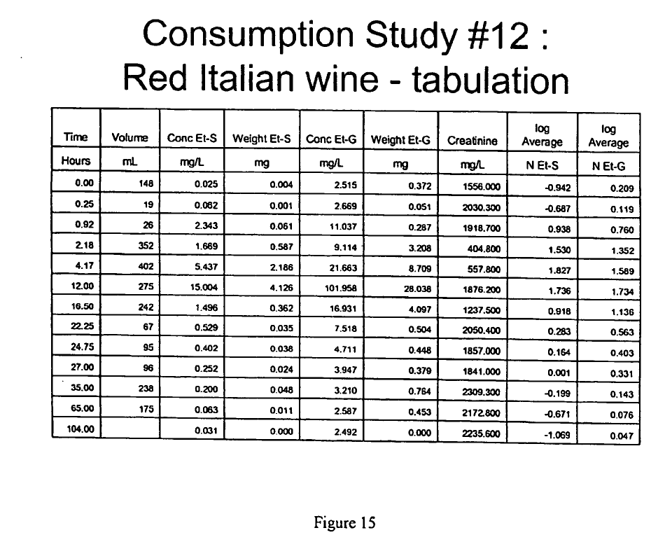

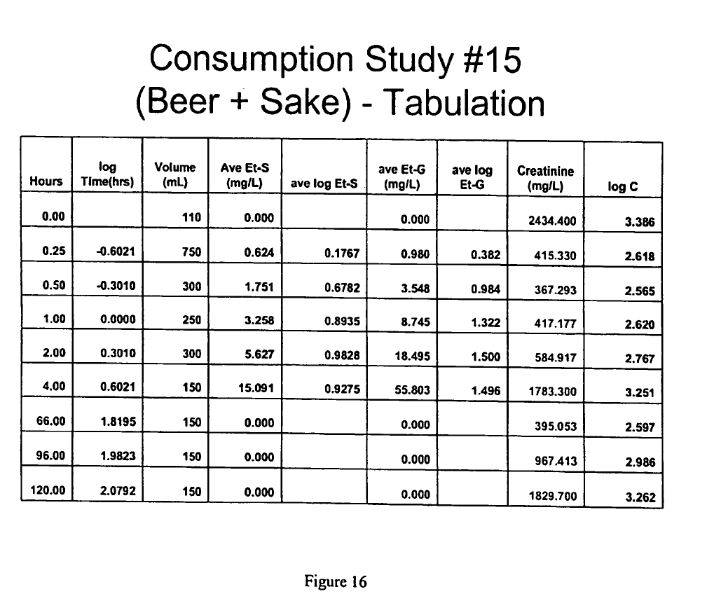

As shown in FIGS. 15 and 16, his creatinine concentration and volume of urination (which affects concentration in the sample greatly) varied during the course of this study, thus indicating the importance of normalization.

This example showed that following consumption of alcoholic beverages it is possible to measure the quantity of the metabolites of ethanol, ethyl glucuronide and ethyl sulfate in the urine as an indicator of alcohol consumption in at least 20 hours after consumption. Various beverages and volunteers were tested. The effect of inadvertent alcohol consumption (e.g. from cough syrup or food) was evaluated and found to be quite insignificant. The effect of normalization to urinary volume and thickness of urine was demonstrated and shown to produce good results.

What is claimed is:

1. A method for quantifying and normalizing at least one product of ethanol metabolism in a sample, said sample comprising creatinine, said method comprising:
(i) adding a predetermined amount of at least one internal standard to the sample;
(ii) adding deuterated creatinine to the sample;
(iii) detecting and measuring the at least one product of ethanol metabolism, the at least one predetermined amount of internal standard in the sample, the deuterated creatinine, and the creatinine;
(iv) quantifying the amount of the at least one product of ethanol metabolism in the sample using the measurement of the at least one internal standard;
(v) quantifying the amount of the creatinine in the sample using the measurement of the deuterated creatinine; and
(vi) normalizing the quantity of the at least one product of ethanol metabolism using the measurement of the creatinine.

2. The method according to claim 1 wherein the sample is urine.

3. The method according to claim 1 wherein the sample is saliva.

4. The method according to claim 1 wherein the sample is blood or plasma.

5. The method according to claim 1 wherein the sample is obtained from a mammal.

6. The method according to claim 4 wherein the mammal is a human.

7. The method according to claim 1 wherein the detecting and measuring is performed by a mass spectrometer.

8. The method according to claim 6 wherein the mass spectrometer comprises a triple quadrupole.

9. The method according to claim 1 wherein the at least one product of metabolism is ethyl glucuronide.

10. The method according to claim 8 wherein the at least one internal standard is deuterated ethyl glucuronide.

11. The method according to claim 1 wherein the at least one product of metabolism is ethyl sulphate.

12. The method according to claim 10 wherein the at least one internal standard is deuterated ethyl sulphate.

13. The method according to claim 1 wherein the sample can be diluted before addition of the at least one internal standard.

14. The method according to claim 1 wherein the method is automated.

15. Use of the method according to claim 1 to predict the time and level of alcohol in a source.

16. The use according to claim 14 wherein the source is a mammal .

17. Use of the method according to claim 1 to monitor alcohol in a source.

18. The use according to claim 16 wherein the source is a mammal.

19. A system for monitoring ethanol metabolism in a source using a mass spectrometer to analyze a sample from the source, said sample comprising creatinine, indicative of the physical state of the source, said system comprising a controller adapted to:
(i) automatically dilute the sample by a predetermined amount at least once;
(ii) add a predetermined amount of an internal standard to the at least one diluted sample;
(iii) add deuterated creatinine to the sample;
(iv) detect and measure at least one product of ethanol metabolism, the at least one internal standard in the sample, the deuterated creatinine, and the creatinine;
(v) quantify the amount of the at least one product of ethanol metabolism in the sample using the measurement of the at least one internal standard;
(vi) quantify the amount of creatinine in the sample using the measurement of the deuterated creatinine; and
(vii) normalize the quantity of the at least one product of ethanol metabolism using the measurement of the creatinine.

20. The system according to claim 18 wherein the source is a mammal.

21. The system according to claim 19 wherein the source is a human.

22. The system according to claim 18 wherein the mass spectrometer comprises a triple quadrupole.

23. The system according to claim 18 wherein the sample is urine.

24. The system according to claim 18 wherein the sample is saliva.

25. The system according to claim 18 wherein the sample is blood/plasma

26. The system according to claim 18 wherein the at least one product of ethanol metabolism is ethyl glucuronide.

27. The system according to claim 24 wherein the at least one internal standard is deuterated ethyl glucuronide.

28. The system according to claim 18 wherein the at least one product of ethanol metabolism is ethyl sulphate.

29. The system according to claim 26 wherein the at least one internal standard is deuterated ethyl sulphate.

30. The system according to claim 18 wherein the sample is diluted before addition of the at least one internal standard.

31. A kit for quantifying and normalizing at least one product of ethanol metabolism in a sample, said sample comprising creatinine, said kit comprising at least one of the following: a sample, a deuterated internal standard, a calibration standard, a quality control check, instructions, and combinations thereof.
